(19) United States
(12) Patent Application Publication
(10) Pub. No.: US 2020/0075158 A1
(43) Pub. Date: Mar. 5, 2020

(54) SYSTEM AND METHOD FOR ACQUIRING AND DECODING PATIENT CLINICAL INFORMATION USING OFF THE SHELF DEVICES

(71) Applicant: COHERE-MED SOLUTIONS PRIVATE LIMITED, Bangalore (IN)

(72) Inventors: Srikanth MUTHYA, Bangalore (IN); Renjith NAIR, Bangalore (IN)

(73) Assignee: COHERE-MED SOLUTIONS PRIVATE LIMITED, Bangalore (IN)

(21) Appl. No.: 16/327,766

(22) PCT Filed: Apr. 26, 2018
(86) PCT No.: PCT/IN2018/050254

§ 371 Date: Feb. 22, 2019

(30) Foreign Application Priority Data
Feb. 16, 2018 (IN) 201841005955

(51) Int. Cl. G16H 40/40; G16H 10/60

(52) U.S. Cl. CPC G16H 40/40; G06F 15/16; G16H 10/60

(57) ABSTRACT

A system to acquire and decode patient clinical information is provided. The system includes an off-the-shelf device. The off-the-shelf device includes at least a processor and memory communicatively coupled to the processor and a low power, long range wireless transmission means communicating with a medical device through one of its ports. The memory includes a plurality of modules for execution by the processor, wherein the modules are configured at least for detecting information about a medical device it is communicatively coupled to; acquiring patient clinical information from the medical device; transforming the patient clinical information into an open standard format; transmitting the transformed patient clinical information securely over a network using a low power, long range wireless protocol. The system also includes a central server configured for receiving the transmitted data for storing and analysis.

### FIELD OF TECHNOLOGY

The present disclosure generally relates to a system and a method to acquire and decode medical device data in the field of health informatics and more particularly to a system and a method for acquiring and decoding patient clinical information from a plurality of medical devices with minimal infrastructural components and accessories and in a secure manner.

### BACKGROUND

It is not new, that the volume of data is practically exploding day by day. Given the amount of massive data explosion happening, more data has been created in the past five years than in the entire previous history of the human race. Even the healthcare industry has become more and more of a data-driven. It has been observed, that there has been a trend towards making medical records available via the internet to make it easier and more efficient for access for patients and also medical experts. Unfortunately data is quite complex when it comes to healthcare. Moreover, the data with respect to health care is scattered and challenging to acquire.

Reliability of patient clinical information obtained through several medical devices used in acute care setting of patients, such as Intensive Care Units (ICU), Operation Theatres (OT), Emergency Care, Triage and the like is important. These are areas where industrial grade life support devices play a vital role and even data transmission needs to be reliable. The data generated by medical devices is large and is continuous. Furthermore, in healthcare, interoperability, which is a challenge, is the ability of different medical devices and software applications to communicate, exchange data, and use the data that has been exchanged. However, data obtained by such medical devices is currently not often interoperable.

In a scenario where a patient needs acute attention, information is key and time is of the essence. A care giver needs to focus his or her attention towards a patient within a fraction of a second. Given that the number of patients needing care is expected to grow across the world, it is humanly impossible to have care givers spend the necessary time with every patient. Moreover, data gets generated from several sources. Comprehending these in a concise manner and making decisions could result in life and death situations for patients. In an ICU or OT, data comes in from various sources such as medical devices to which the patient is connected. For example, the various sources or information systems include Electronic Medical Records (EMR), Hospital Information Systems (HIS), Laboratory Information Systems (LIS), Radiology Information Systems (RIS), Picture Archival and Communication Systems (PACS) etc. In most cases, the data is lying in silos in these information systems and is collected and collated manually. The data output from the medical devices include waveforms, physiological parameters and alarms. However, in existing solutions unless proprietary hardware, middleware or application software is used, there are practically no means to get all kinds of above data. Moreover, such solutions are primarily provided by the equipment manufacturers who control the entire ecosystem.

It is estimated that the information systems known in the art generally provide, only 40 to 60% of data that can be automated and delivered by medical devices to which patients are connected. There are ways and means today provided by independent third party providers and original medical device manufacturers, where data acquisition can be automated from the medical devices. However, these are cost ineffective and rely on large infrastructural requirements. This forms a barrier to automate and digitise data to produce meaningful results. Moreover, for acquiring patient clinical information from these medical devices, there is a need to follow protocols which are proprietary in nature. This poses further problems to standardise data that can be consumed by any information system.

Furthermore, systems known in the art generally provide two ways in which data can be acquired from medical devices and decoded with existing technologies. One is technology provided by medical device manufacturers and the other third party providers (just a handful of them) who build medical device interfaces. Most large information technology (IT) companies leverage these technologies to interface medical devices with Electronic Medical Records (EMR) and other information systems at hospitals. These are prevalent mainly in developed markets and are not cost effective.

### SUMMARY

In order to solve at least some of the above mentioned problems, there exists a long felt need for a system and a method for acquiring and decoding patient clinical information obtained from a plurality of medical devices with minimal infrastructural components and accessories and in a secure manner. A system and a method is needed that will seamlessly acquire the patient clinical information from a plurality of industrial grade medical devices or machines, transform the acquired patient clinical information in a standardised manner which is meaningful for further analysis. A system and a method is needed that unifies the data irrespective of the vendor who has manufactured creating a truly interoperable information exchange.

This summary is provided to introduce a selection of concepts in simple manners that are further described in the detailed description of the disclosure. This summary is not intended to identify key or essential inventive concepts of the subject matter nor is it intended to determine the scope of the disclosure.

Briefly, according to an exemplary embodiment, a system to acquire and decode patient clinical information is provided. The system includes an off-the-shelf device. The off-the-shelf device includes at least a processor and a memory communicatively coupled to the processor and a low power, long range wireless transmission means, communicatively coupled to a medical device through one of its ports. The memory has been loaded with a plurality of modules for execution by the processor, wherein the plurality of modules are configured at least for detecting one or more information about a medical device it is communicatively coupled to; acquiring patient clinical information, depending on the detected one or more information, from the medical device; transforming the patient clinical information into an open standard format; transmitting the transformed patient clinical information securely over a network using a low power, long range wireless protocol. The system also includes a central server configured for receiving the transmitted data for storing and analysis.

Briefly, according to an exemplary embodiment, a method for acquiring and decoding patient clinical information is provided. The method includes detecting one or more information, about a medical device it is communicatively coupled to. The method also includes acquiring patient clinical information depending on the detected one or more information, from the medical device. The method further includes transforming the patient clinical information into an open standard format. In addition, the method includes transmitting the transformed patient clinical information securely over a network using a low power long range wireless protocol. In addition, the method includes receiving the transmitted data for storing and analysis.

The summary above is illustrative only and is not intended to be in any way limiting. Further aspects, exemplary embodiments, and features will become apparent by reference to the drawings and the following detailed description.

### BRIEF DESCRIPTION OF THE FIGURES

These and other features, aspects, and advantages of the exemplary embodiments can be better understood when the following detailed description is read with reference to the accompanying drawings in which like characters represent like parts throughout the drawings, wherein:

FIG. 1 is a block diagram of one embodiment of a system comprising a plurality of off-the-shelf devices configured for acquiring patient clinical information seamlessly from a plurality of medical devices or machines, transforming the acquired patient clinical information into standardised manner and transmitting the data securely to a central server for further analysis, according to an embodiment of the present disclosure;

FIG. 2 illustrates the exploded view of the off-the-shelf devices of the system of FIG. 1, according to an embodiment of the present disclosure;

FIG. 3 illustrates the example implementation of the plurality of off-the-shelf devices interconnected with a plurality of medical devices that monitors a patient, and configured for acquiring patient clinical information from the medical devices, transforming the acquired patient clinical information in standardised manner and transmitting the data securely for further analysis, according to an embodiment of the present disclosure; and

FIG. 4 is a flow chart illustrating a method for acquiring patient clinical information seamlessly from a plurality of medical devices or machines, transforming the acquired patient clinical information into standardised manner and transmitting the data securely to a central server for further analysis, according to an embodiment of the present disclosure.

Further, skilled artisans will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present invention so as not to obscure the figures with details that will be readily apparent to those of ordinary skill in the art having the benefit of the description herein.

### DETAILED DESCRIPTION

For the purpose of promoting an understanding of the principles of the invention, reference will now be made to the embodiments illustrated in the figures and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

The terms “comprises”, “comprising”, or any other variations thereof, are intended to cover a non-exclusive inclusion, such that a process or method that comprises a list of steps does not comprise only those steps but may comprise other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components. Appearances of the phrase “in an embodiment”, “in another embodiment” and similar language throughout this specification may, but do not necessarily, all refer to the same embodiment.

Unless otherwise defined, all technical and scientific terms used herein have the same meaning as commonly understood by one of ordinary skill in the art to which this invention belongs. The system, methods, and examples provided herein are illustrative only and not intended to be limiting.

In addition to the illustrative aspects, exemplary embodiments, and features described above, further aspects, exemplary embodiments of the present disclosure will become apparent by reference to the drawings and the following detailed description.

The key feature of this disclosure is making use of off-the shelf devices to create a network for acquiring medical data for storage and further processing. The off-the-shelf device comprises at least a processor, a memory communicatively coupled to the processor, and a low power, long range wireless transmission means. The primary intent of these off-the-shelf devices is easy creation of internet of things (IoT). These devices are primarily meant for acquiring signals from one or more sensors and process them for use in an Internet of Things. In this disclosure, such devices have been configured to acquire, decode and transform data from a diverse set of medical devices and process them.

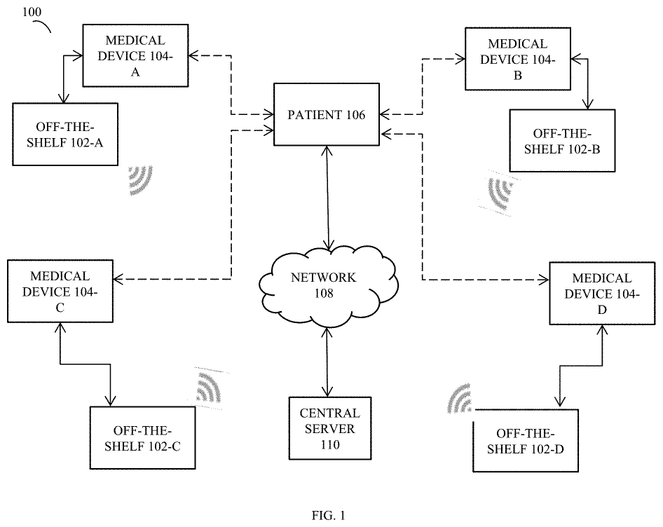

FIG. 1 is a block diagram of one embodiment of a system 100 comprising a plurality of off-the-shelf devices configured for acquiring patient clinical information seamlessly from a plurality of medical devices or machines, transforming the acquired patient clinical information into standardised manner and transmitting the data securely to a central server for further analysis, according to an embodiment of the present disclosure. In particular, the system 100 illustrates the plurality of off-the-shelf devices 102-A-102-N, each one connected to one of a plurality of medical devices 104-A-104-N. The plurality of medical devices 104-A-104-N are intended to monitor a patient and take the measurements of physiological parameters and the like of a patient 106. The off-the-shelf devices 102-A-102-N are configured to acquire the data through the plurality of medical devices 104-A-104-N and transmit the acquired patient clinical information to the central server 110 via a network 108.

The system 100 makes use of off-the-shelf devices, (102-A-102-N) using minimal infrastructural needs to acquire data from the plurality of medical devices (104-A-104-N). Each off-the-shelf device (102-A-102-N) as illustrated in FIG. 1 connects communicatively to one of the medical devices (104-A-104-N) with a physical cable designed specifically for the particular medical device in question. This communicative coupling to a medical device (104) is through one of the ports of the medical device.

The off-the-shelf device comprising at least a processor, a memory communicatively coupled to the processor, and a low power, long range wireless transmission means is adapted for the purpose of creating a patient clinical information system as follows. The memory has been loaded with a plurality of modules for execution by the processor, wherein the plurality of modules are configured at least for detecting one or more information about a medical device it is communicatively coupled to, acquiring patient clinical information, depending on the detected one or more information, from the medical device, transforming the patient clinical information into an open standard format, transmitting the transformed patient clinical information securely over a network using a low power, long range wireless protocol. The system 100 also includes the central server 110 configured for receiving the transmitted data for storing and analysis.

The off-the-shelf device (102-A-102-N) is configured to automatically detect the medical device (104) and begin communication with the medical device (104) based on the proprietary commands provided by the device manufacturer. The off-the-shelf devices (102-A-102-N) as illustrated in FIG. 1, when connected communicatively to the medical device (104-A-104-N), is configured to automatically detect the details of the medical device. The detection involves four key elements such as a port sniffer, packet compliance, protocol assessment and data distribution. The port sniffer is an automated script built at the kernel level to sniff any external devices connected through any of the available ports such as USB, Serial and other general purpose IO (GPIO) interfaces available through Serial Peripheral Interface (SPI) and Inter-integrated Circuit (I2C) Protocol. Upon receipt of the port being open, a pre-configured byte size is sniffed to check packet compliance from a repository pre-loaded into as a part of software in the off-the-shelf device (102). If the medical devices (104) is detected and identified, further more number of bits are read into a stream to assess the protocol. This initial level of decoding enables to conform to standards and ensures clean data as per manufacturer specifications. A global flag is then set to acknowledge that the medical device (104) is ready to transmit data into the data distribution system post which all the other processes begin.

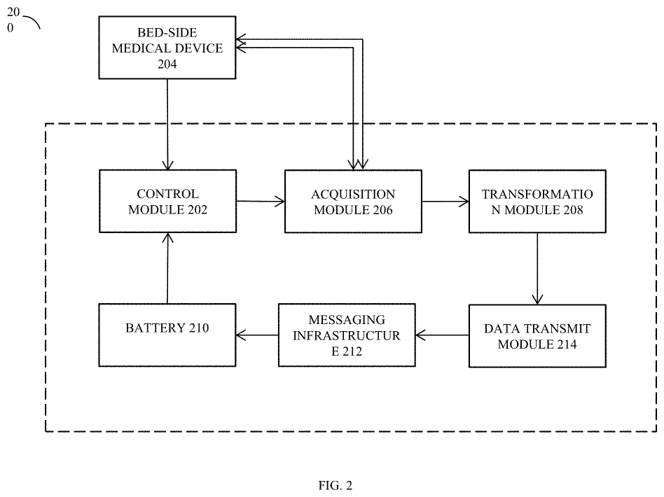

The off-the-shelf devices (102-A-102-N) are configured to acquire patient clinical information which is in the form of waveform, physiological parameters and alarms from the medical devices (104-A-104-N) without using any middleware or an application. In one embodiment, the off-the-shelf devices (102-A-102-N) may be Commercial-off-the-shelf (COTS) software and services that are built and delivered usually from a third party vendor. COTS can be obtained and operated at a lower cost over in-house development, and provide increased reliability and quality over custom-built software as these are developed by specialists within the industry and are validated by various independent organisations, often over an extended period of time. The operation of the off-the-shelf devices (102-A-102-N) is explained in detail below in FIG.2.

The medical devices 104-A-104-N include but are not limited to, single or multi-parameter patient monitor used to record vital signs of patients, ventilators used to provide therapy for breathing, infusion/syringe pumps for fluid & injections, blood gas analysers, anaesthesia machines, heart lung machine, dialysis machine or combinations thereof. The medical devices 104-A-104-N primarily are of industrial grade used in Intensive Care Unit (ICU), Emergency Care, Triage, Operation Theatre (OT), birthing suites in a hospital, home ICU and the like. In one example, the medical devices 104-A-104-N may be a third party patient multi-parameter vital sign monitor, a third party patient invasive ventilator and a third party syringe or infusion pump.

The patient clinical information is acquired by the off-the-shelf devices (102-A-102-N) from the medical devices (104-A-104-N) at a very high sampling rates configurable up to 1 second and transformed into a message with standard formats. In one embodiment, the message contents are syntactically and semantically structured such that the message is interoperable. The data structure of the message can be also be transformed into standard formats like HL7 v2.x message structure or HL7 FHIR structure or any desired format. The data structure is transformed using underlying standard based structure (taking care of the syntactic structure) like HL7 v2 message structure/HL7 FHIR data model. In one embodiment, the standardised, multi-lingual vocabulary of clinical terminology (like SNOMED-CT, LOINC) to cover semantic interoperability is utilized for data transformation.

Further, the patient clinical information collected from the off-the-shelf devices (102-A-102-N) including the clinical data and the meta-data describing the device along with the identification information is included in the message to be transmitted to the central server 110 via a wireless network 108. In one example, the message may also include further identification information on the care setting—hospital department (ICU), and clinic identification. Furthermore, the data acquired by the off-the-shelf devices (102-A-102-N) from the medical devices (104-A-104-N) is transmitted to the central server 110. The central server 110 is a cloud server. The central server 110 may be a wireless end point hosting a tablet or a computer.

A secured layer consisting of an end-point where the messaging packets need to be delivered is built into off-the-shelf devices (102-A-102-N) which is then transmitted on a Secured Socket Layer (SSL) layer onto the central server 110 (cloud) for storage, taking necessary action, and further analysis and the like. The central server 110 as described in the present disclosure can be implemented to perform additional functions, even though not explicitly mentioned in the present disclosure.

In one example embodiment, LoRa technology may be implemented as the network 108 for data transmission from the off-the-shelf devices (102-A-102-N) to the central server 110. The transmission using LoRa technology allows low power and long range capabilities. Due to the low power consumption of wireless transmission, the off-the-shelf devices (102-A-102-N) gets an advantage of executing larger number of instruction set to manage data transmission consistently at a sampling frequency of 1 KHz, with less than 4.5 mA power consumption. In one example, the LoRa stands for Low-Power Wide-Area Network (LPWAN). In one example, the LoRa is the wireless technology mainly targeted for M2M, IoT networks and Commercial-off-the-shelf (COTS). This technology enables public or multi-tenant networks to connect multiple applications running in the same network. In one example, the low-power wireless networks are a key enabler for the Internet of Things (IoT), but familiar options such as Bluetooth, ZigBee, Wi-Fi, or cellular, lack an acceptable combination of extended range and battery life. To address this, new sub-GHz specifications are being offered, one of which is LoRaWAN which is implemented by the system 100 of the present disclosure. LoRaWAN can achieve a 15 km range at power consumption levels low enough to enable a ten year battery life. Further, the availability of an off-the-shelf development kit lets designers quickly bring up a complete LoRaWAN network application with minimal effort.

For example, LoRa technology developed by Semtech, may be implemented for transmission of transformed patient clinical information securely to the central server 110. The LoRaWAN MAC protocol is designed to support IoT applications with different requirements for downlink communications from a LoRaWAN gateway to an end device. The LoRaWAN gateway form the bridge between devices and the Things network. For example, the off-the-shelf devices (102-A-102-N) may use low power networks like LoRaWAN to connect to the gateway, while the gateway uses high bandwidth networks like WiFi, Ethernet or Cellular to connect to the Things network.

The system 100 has enhanced security and a way to communicate its status to monitoring stations to ascertain its state. Patient clinical information thus acquired using the off-the-shelf devices (102-A-102-N) of system 100 and stored in the central server 110 also can be configured to a high resolution that can be used for further research purposes.

FIG. 2 illustrates an exploded view 200 of the off-the-shelf device of system 100 of FIG. 1, according to an embodiment of the present disclosure. In particular, the off-the-shelf device includes a control module 202, an acquisition module 206, a transformation module 208, a data transmit module 210, a messaging infrastructure 212, a battery 214. The off-the-shelf device is connected to a bed-side medical device 204.

In one embodiment, the off-the-shelf device 200 includes at least a processor and a memory communicatively coupled to the processor and a low power, long range wireless transmission means, communicatively coupled to a medical device 204 through one of its ports. In the same embodiment, the memory is loaded with a plurality of modules for executing by the processor. The plurality of modules are the control module 202, the acquisition module 206, the transformation module 208, the data transmit module 210, the messaging infrastructure 212.

The modules that are stored in the memory of the off-the-shelf device has been deployed a very low foot print off-the-shelf hardware running SoC (off-the-shelf device as shown in FIG. 2) for replacing proprietary hardware needed to be installed by the device manufacturer or a third party provider. The off-the-shelf device interconnected with the medical devices is integrated with a LoRa transmitter to make use of the long range and low power advantage.

In one embodiment, the control module 202 existent in the off-the-shelf device 200 is powered ON, by directly using the 5V USB port available in the medical device 204. The power is enough to drive the off-the-shelf device (200), and the wireless radio built and driven as a part of SoC design. The control module 202 once powered ON automatically detects the medical device 204 connected and loads the appropriate configuration for data acquisition. The control module 202 also auto configures wireless network available in the hospital for outbound traffic. Further, the control module 202 also holds a state machine so that at any given point of time the state of the entire off-the-shelf device (200) is known. An ‘Alive’ message is frequently sent to ensure that the off-the-shelf device (200) issues are escalated immediately so that appropriate actions can be taken.

The acquisition module 206 is configured to acquire patient clinical information which is a raw data output in the form of waveform, physiological parameters and alarms from the medical device 204 without using any middleware or an application. The acquisition module 206 then initiates data transfer based on the proprietary protocol spec provided by the medical device (204) manufacturer. The module or a firmware is written in a small footprint native language that provides assistance to run on minimalistic SoC based board using not more than 500 MB of runtime memory space.

The data transformation module 208 is configured for transforming the acquired patient clinical information into an open standards such as HL7 (Health Level 7) which is ultimately stored into a FHIR (Fast Health Interoperable Resource) repository (not shown). The message contents are well defined in a syntactic structure that allows such conversions between standard or non-standard formats to be feasible. The data contents are also semantically interoperable with use of standardised, multilingual vocabulary of clinical terminology. The patient clinical information acquired from the medical device 204, particularly the clinical data is encapsulated in the message and additional information required for unique identification of the medical device 204 and the care setting is included.

The data transformation module 208 is communicatively coupled to the messaging infrastructure 212 via the data transmit module 214. In one embodiment, the data transmit module 214 is a standard or proprietary communication interface. Examples of communication interface 112, include but are not limited to, HL7 interface, XML interface or combinations thereof.

The data thus transformed is transmitted further using data transmit module 210 over a double layered security protocol using SSL towards an encrypted end point that is in-turn is in a secured environment. The secured layer is provided by the messaging infrastructure 212, which also ensure a high quality of service so that the packet drop is kept to minimal due to LoRa. In one example embodiment, the LoRa (Low-Power Wide-Area Network (LPWAN)) is a modulation technique based on spread-spectrum techniques and a variation of Chirp Spread Spectrum (CSS), which provides significantly longer range than the competing technologies. The LoRa modulation is the physical layer of LoRaWAN, which is a MAC protocol for a high capacity long range and Low Power Wide Area Networks (LPWAN). The LoRa Alliance has tried to make the LoRaWAN very secure by adding three different layers of security such as Unique Network key to ensure security on network level, Unique Application key to ensure end to end security on application level and Device specific key.

A battery 214 internal to the architecture helps manage this off-the-shelf device for over 10 hours due to low power consumption and fewer instruction set that the connect runs on.

In cases, where the medical device 204 does not support USB port, the unique adapter which has built-in electrical isolation is built in to draw power from wall socket. A simple cross over cable or a serial cable is used to connect the medical device 204 to off-the-shelf device board. In one embodiment, isolators are used for both data and power. In one example, for data, the isolator used is Silicon Lab chipset SI8621, a dual channel digital isolator. Isolation is rated as high as 5 kV. Typically the ratings for basic isolation is only 1.5 kV. In another example, for power, the isolator used is Murata power solutions, isolated dc-dc converter rated at 3 kV. Both are Underwriters Laboratories (UL) recognised components to drive safety.

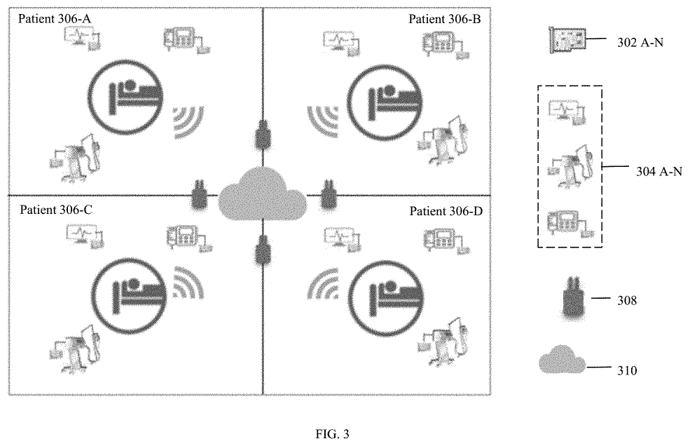

FIG. 3 illustrates the example implementation of the plurality of off-the-shelf devices interconnected with plurality of medical devices that monitors a patient, and configured for acquiring patient clinical information from the medical devices, transforming (decoding) the acquired patient clinical information into standardised manner and transmitting the data securely for further analysis, according to an embodiment of the present disclosure. In particular, FIG. 3 illustrates a plurality of off-the-shelf devices 302-A-302-N interconnected to a plurality of medical devices 304-A-304-N. The plurality of medical devices 304-A-304-N are being implemented to monitor and take the results of a patient 306-A-306-N. The off-the-shelf devices 302-A-302-N are configured to acquire the patient clinical information through the plurality of medical devices 304-A-304-N, transform the acquired patient clinical information into a standard format and transmit the acquired patient clinical information to the central server 310 via a network 308.

Referring to FIG. 3, a four bedded ICU is shown wherein four patients, patient 306-A, patient 306-B, patient 306-C and patient 306-D are being monitored and treated from various medical devices 304-A-304-N. Most medical devices today have the capability to export data in a format which is either standard or, proprietary based protocol. Today, the proprietary nature of the protocol makes hospitals to purchase additional accessories at a significant cost, again mostly provided by the respective device manufacturers to ensure data is exported for clinical use. Moreover, the data exported with the help of device manufacturer accessory would produce data of limited samples, prohibiting further clinical research and discovery of unknown patient degradation process.

The off-the-shelf devices 302-A-302-N, loaded with a unique software connects to the medical (bedside) devices 304-A-304-N. The off-the-shelf devices 302-A-302-N acquires the patient clinical information from the medical (bedside) devices 304-A-304-N in the proprietary format. The off-the-shelf devices 302-A-302-N then transforms the acquired patient clinical information which is in a particular format to a standardised format. The data in a standardised format is transmitted wirelessly (For example, LoRa) by the off-the-shelf devices 302-A-302-N. The central server 310 receives the acquired patient clinical information in a standard format, stores it, and analyses it to raise alarms and so on based on different criteria.

The system 100 of the present disclosure as described above comes with the fact that it breaks the proprietary nature of protocols into standardised means. Secondly, the cost of the implementation of the system 100 is substantially lower than those available today. This is due to the fact that the system 100 takes lesser footprint to run. In addition, system 100 performs several other tasks such as communicating on a wireless secured socket layer built on LoRa (Low Power Wide Area Network technology). Most solutions today rely on a standard wireless or an Ethernet based infrastructure. In this case, data is designed to be transmitted via LoRa technology which leads to a minimal infrastructure dependent architecture. The system 100 of the present disclosure does not need a proprietary hardware and setup with large infrastructure to acquire and decode patient clinical information, on the other hand the system 100 operates more on off-the-shelf components using minimal infrastructural needs, thereby reducing cost and hence provides an economically significant advantage.

Another advantage of the implementation of the system 100 includes workforce productivity through automation of machine data. Furthermore, the medical errors are reduced thereby reducing length of stay. In addition, the system 100 leads to cost reductions and improvement in revenue per occupied bed in ICU. Moreover, the system 100 results in asset utilisation and effectiveness further resulting in overall hospital growth into other geographies. In addition, the system 100 provides, deep learning insights on clinical data for research purposes.

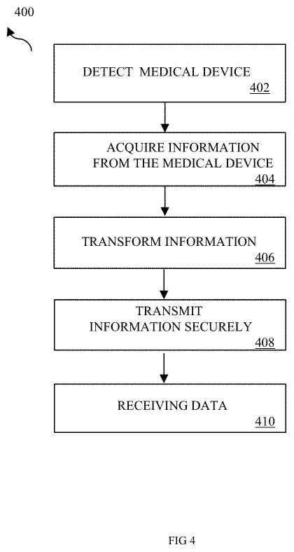

FIG. 4 is a flow chart illustrating a method 400 for acquiring patient clinical information seamlessly from a plurality of medical devices or machines, transforming the acquired patient clinical information into standardised manner and transmitting the data securely to a central server for further analysis, according to an embodiment of the present disclosure. FIG. 4 will be described from the perspective of a processor that is configured to execute computer-readable instructions to carry out the functionalities of the components of the off-the-shelf device 200 as shown in FIG. 2. Each step is described in detail below.

At step 402, one or more information is detected, about a medical device it is communicatively coupled to. An off-the-shelf device connects to the medical devices with a physical cable designed specifically for the particular medical device in question. The off-the-shelf device is configured to automatically detect the medical device and begin communication with the medical device based on the proprietary commands provided by the device manufacturer.

At step 404, patient clinical information is acquired, depending on the detected one or more information, from the medical device. The acquisition module 206 of FIG. 2 is configured to acquire patient clinical information which is a raw data output in the form of waveform, physiological parameters and alarms from the medical device without using any middleware or an application.

At step 406, the patient clinical information is transformed into an open standard format. The data transformation module 208 of FIG. 2 is configured for transforming the acquired patient clinical information into an open standards such as HL7 (Health Level 7) which is ultimately stored into a HL7 FHIR (Fast Health Interoperable Resource) repository (not shown). The message contents are well defined in a syntactic structure that allows such conversions between standard or non-standard formats to be feasible. The data contents are also semantically interoperable with use of standardised, multilingual vocabulary of clinical terminology. The patient clinical information acquired from the medical device, particularly the clinical data is encapsulated in the message and additional information required for unique identification of the medical device and the care setting is included.

At step 408, the transformed patient clinical information is transmitted securely over a network using a low power long range wireless protocol. The data thus transformed further is transmitted using data transmit module 210 of FIG. 2 over a double layered security protocol using SSL towards an encrypted end point that is in-turn is in a secured environment. The secured layer is provided by the messaging infrastructure 212 of FIG. 2, which also ensure a high quality of service so that the packet drop is kept to minimal due to LoRa. In one example embodiment, the LoRa (Low-Power Wide-Area Network (LPWAN)) is a modulation technique based on spread-spectrum techniques and a variation of Chirp Spread Spectrum (CSS), which provides significantly longer range than the competing technologies. The LoRa modulation is the physical layer of LoRaWAN, which is a MAC protocol for a high capacity long range and Low Power Wide Area Networks (LPWAN). The LoRa Alliance has tried to make the LoRaWAN very secure by adding three different layers of security such as Unique Network key to ensure security on network level, Unique Application key to ensure end to end security on application level and Device specific key.

At step 410, the transmitted data is received for storing and analysis. The data which is the acquired patient clinical information is received by the central server 110 (cloud) of FIG. 1 for storage, taking necessary action, and further analysis and the like. The patient clinical information thus received in standard format is stored in the central server 110 and also can be configured to a high resolution that can be used for further research purposes.

While specific language has been used to describe the disclosure, any limitations arising on account of the same are not intended. As would be apparent to a person skilled in the art, various working modifications may be made to the method in order to implement the inventive concept as taught herein. The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

1. A system to acquire and decode patient clinical information, the system comprising:
an off-the-shelf device comprising at least a processor and a memory communicatively coupled to the processor and a low power, long range wireless transmission means, communicatively coupled to a medical device through one of its ports, wherein the memory has been loaded with a plurality of modules for execution by the processor, wherein the plurality of modules are configured at least for:
detecting one or more information about a medical device it is communicatively coupled to;
acquiring patient clinical information, depending on the detected one or more information, from the medical device;
transforming the patient clinical information into an open standard format;
transmitting the transformed patient clinical information securely over a network using a low power, long range wireless protocol; and
a central server configured for receiving the transmitted data for storing and analysis.

2. The system of claim 1, wherein the off-the-shelf device is powered by the medical device via the port of the medical device.

3. The system of claim 1, wherein the off-the-shelf device comprises an adapter with built-in galvanic isolation circuit for powering the device from an external power source.

4. The system of claim 1, wherein the off-the-shelf device is a plug and play device and is configured for self-detection of network and initiating acquisition of patient clinical information from the detected medical device.

5. The system of claim 1, wherein the transformed patient clinical information comprises a message with interoperable capabilities by use of standardised, multi-lingual vocabulary of clinical terminology.

6. The system of claim 5, wherein the message further comprises identification information on one or more of a care setting, a hospital department and clinic identification.

7. A method for acquiring and decoding patient clinical information, the method comprising:
detecting one or more information, about a medical device it is communicatively coupled to;
acquiring patient clinical information depending on the detected one or more information, from the medical device;
transforming the patient clinical information into an open standard format;
transmitting the transformed patient clinical information securely over a network using a low power long range wireless protocol; and
receiving the transmitted data for storing and analysis.

8. The method of claim 7, wherein the said steps of detecting, acquiring, transforming and receiving are executed by an off-the-shelf device comprising at least a processor and a memory communicatively coupled to the processor, wherein the memory has been loaded with a plurality of modules for executing by the processor.

9. The method of claim 7, wherein the transformed data comprises a message with interoperable capabilities by use of standardised, multi-lingual vocabulary of clinical terminology.
